(19) United States
(12) Patent Application Publication
(10) Pub. No.: US 2021/0361881 A1
(43) Pub. Date: Nov. 25, 2021

(54) INJECTION DEVICE WITH COMMIT FEATURE

(71) Applicant: Owen Mumford Limited, Woodstock, Oxfordshire (GB)

(72) Inventors: Daniel Garson, Woodstock, Oxfordshire (GB); Andreas Artelsmair, Woodstock, Oxfordshire (GB); Brady King, Woodstock, Oxfordshire (GB)

(21) Appl. No.: 17/278,451

(22) PCT Filed: Sep. 20, 2019
(86) PCT No.: PCT/EP2019/075265

§ 371 Date: Mar. 22, 2021

(30) Foreign Application Priority Data
Sep. 28, 2018 (GB) 1815852.7

(51) Int. Cl. A61M 5/32; A61M 5/20

(52) U.S. Cl. CPC A61M 5/326; A61M 5/2033; A61M 2005/3267; A61M 2005/2073; A61M 2005/2013

(57) ABSTRACT

An injection device comprising a body for receiving a syringe, a firing mechanism comprising a plunger configured to be axially displaced in a forward direction within the body and a driver system for driving the plunger forwards upon activation of the injection device, and a lockout shroud telescopically coupled to a forward end of the body so that it projects from the forward end at least immediately prior to activation in order to shroud a syringe needle. The device further comprises a biasing member for biasing the lockout shroud in a forward direction relative to the body, the lockout shroud being coupled to the firing mechanism such that a first predefined rearward movement of the shroud relative to the body releases the driver system to drive the plunger forward, and a lockout shroud inhibitor configured to provide increased resistance to rearward movement of the lockout shroud following a second predefined rearward movement of the lockout shroud, less than said first predefined rearward movement.

### TECHNICAL FIELD

The invention relates to injection devices for delivering a fluid substance to a user and in particular to auto-injectors for delivering the fluid under a force applied by a drive system.

### BACKGROUND

Injection devices are used for the convenient administration of medicaments to patients. For example, injection devices, which may be auto-injectors, may be used for providing a single metered dose of a medicament. Such devices may be either single use “disposable” devices in which the device is typically provided with a syringe already installed, and which is not user-replaceable, or “reusable” devices which allow the user to replace the syringe when the medicament has been used.

It is noted that whilst the term “syringe” is used herein for clarity and consistency, this term is not intended to be limiting. In some arrangements the syringe may for example be a cartridge (which, for example, may be arranged to receive a disposable needle) or other medicament container. In some arrangements the syringe/cartridge/medicament container may be formed integrally with the (or part of the) injection device.

Injection devices may be provided in the form of an auto-injector device comprising a firing mechanism that is arranged to deliver the fluid from the syringe automatically under the force of a drive system, such as a drive spring. Auto-injectors may also comprise an insertion mechanism to displace the syringe within a housing of the injection device to cause needle penetration. The delivery arrangement generally acts via a plunger which includes a plunger and may also include or engage a piston (also referred to as a “bung”) which is slidably provided within the syringe. Whilst in some auto-injectors the firing mechanism is activated by means of a finger operated button or trigger located on the rear or a side area of the device body, other devices are activated by pressing a lockout shroud against the skin, where the lockout shroud extends telescopically from a front end of the device body to shield the needle when not in use. Devices with the latter activation mechanism may be easier to use, particularly for users with limited hand and finger movement.

When preparing for an injection, a self-administering user will often seek an area of skin that is most comfortable for the injection. This may involve touching the end of the device against various regions of the skin to identify a comfortable site. This is often referred to as “site roaming”. In the case of devices that are activated by pressing the lockout shroud into the device body, site roaming does give rise to a small risk that the device may be accidentally activated.

### SUMMARY

Methods and apparatus disclosed herein may be arranged to mitigate or solve one or more problems associated with the art, including those mentioned above and/or elsewhere herein.

According to a first aspect of the present invention there is provided an injection device comprising a body for receiving a syringe, a firing mechanism comprising a plunger configured to be axially displaced in a forward direction within the body and a driver system for driving the plunger forwards upon activation of the injection device, and a lockout shroud telescopically coupled to a forward end of the body so that it projects from the forward end at least immediately prior to activation in order to shroud a syringe needle. The device further comprises a biasing member for biasing the lockout shroud in a forward direction relative to the body, the lockout shroud being coupled to the firing mechanism such that a first predefined rearward movement of the shroud relative to the body releases the driver system to drive the plunger forward, and a lockout shroud inhibitor configured to provide increased resistance to rearward movement of the lockout shroud following a second predefined rearward movement of the lockout shroud, less than said first predefined rearward movement.

The lockout shroud inhibitor may provide a reduced resistance after said increased resistance is overcome and prior to activation.

The lockout shroud inhibitor may act between the lockout shroud and the body. It may comprise, for example, at least one pair of mechanically interfering features. The or each pair of mechanically interfering features may comprise a resilient member on one of the lockout shroud and the body and a lip on the other of the lockout shroud and the body, the resilient member engaging the lip at substantially said second predefined position and being configured to flex over the lip when an increased force is applied between the lockout shroud and the body.

Alternatively, the or each pair of mechanically interfering features may comprises a longitudinally extending track on one of the lockout shroud and the body and a feature for engaging and travelling along said track on the other of the lockout shroud and the body, said track comprising a restriction part-way along its length and having sides configured to flex in a transverse direction to enable said feature to pass the restriction when an increased force is applied between the lockout shroud and the body.

The device may comprise a clutch having a substantially fixed axial position within the body and being coupled to the lockout shroud such that said first predefined movement of the lockout shroud rotates the clutch to thereby release the driver system.

The biasing member may be a compression spring coupled between the lockout shroud and the body.

The lockout shroud may be located partially within said body.

### BRIEF DESCRIPTION OF THE DRAWINGS

Exemplary embodiments will be described herein with reference to the accompanying drawings, in which:

FIG. 1 is an exploded view of an auto-injector;

FIG. 2 is a section through an auto-injector with a syringe fitted therein;

FIG. 3 shows a perspective view of an exemplary firing mechanism assembly;

FIG. 4 illustrates a force profile for device activation;

FIG. 5 illustrates an auto-injector incorporating a commit feature with the force profile of FIG. 4, in a stored state;

FIG. 6 illustrates the auto-injector of FIG. 5 with a cap removed;

FIG. 7 illustrates the auto-injector of FIG. 5 with a cap removed;

FIG. 8 illustrates the auto-injector of FIG. 5 with a commit feature engaged;

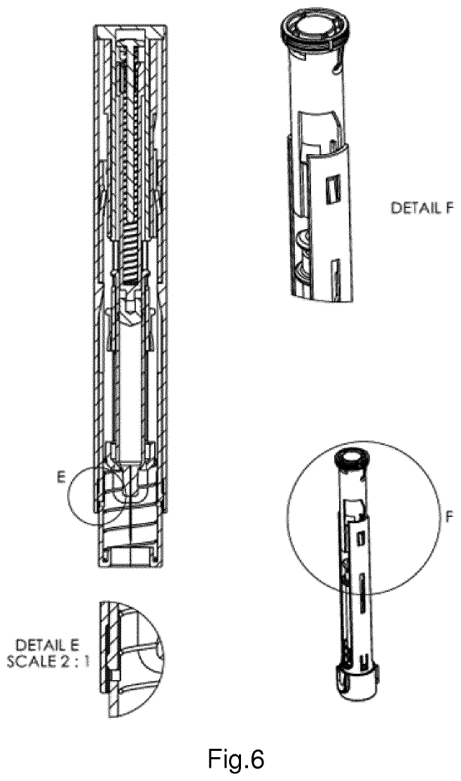

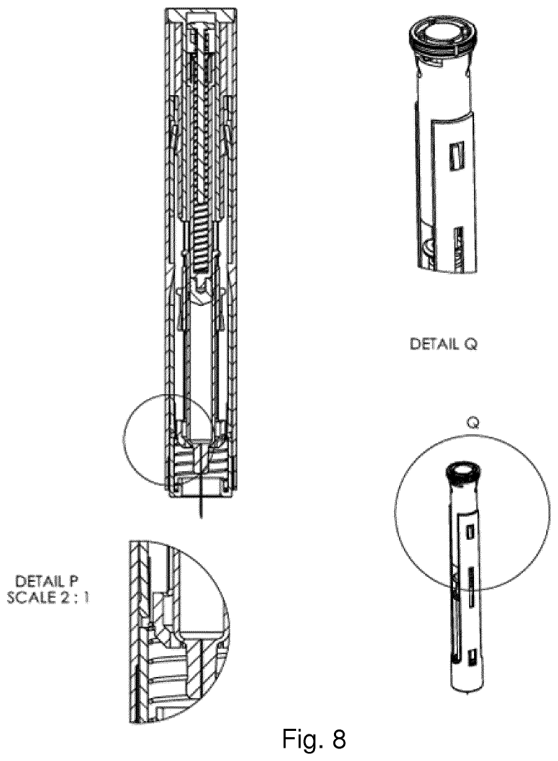

FIG. 7 illustrates the auto-injector of FIG. 5 in an activated state;

FIG. 9 is a perspective view of a lockout shroud of an auto-injector including guide tracks forming part of a commit feature; and

FIG. 10 shows a side view of the lockout shroud of FIG. 9.

### DETAILED DESCRIPTION

In the following embodiments, the terms “forward” and “front” refer to the patient facing end of the injection device or component thereof. In other words, the front end of the injection device is the end proximal to the injection site during use. Likewise, the term “rear” refers to the non-patient end of the injection device assembly or component thereof. In other words, the term “rear” means distant or remote from the injection site during use.

Many features of the exemplary arrangements disclosed herein are described as being “coupled” to other features. This term encompasses any coupling that results in the coupled features moving together in any direction, whether that be on a 1:1 basis or on some geared basis. The term “coupled” also encompasses any one of a connection between features, an abutment of one feature against another and an engagement of one feature with another, and such coupling may be direct or may be indirect, i.e. with a third feature therebetween.

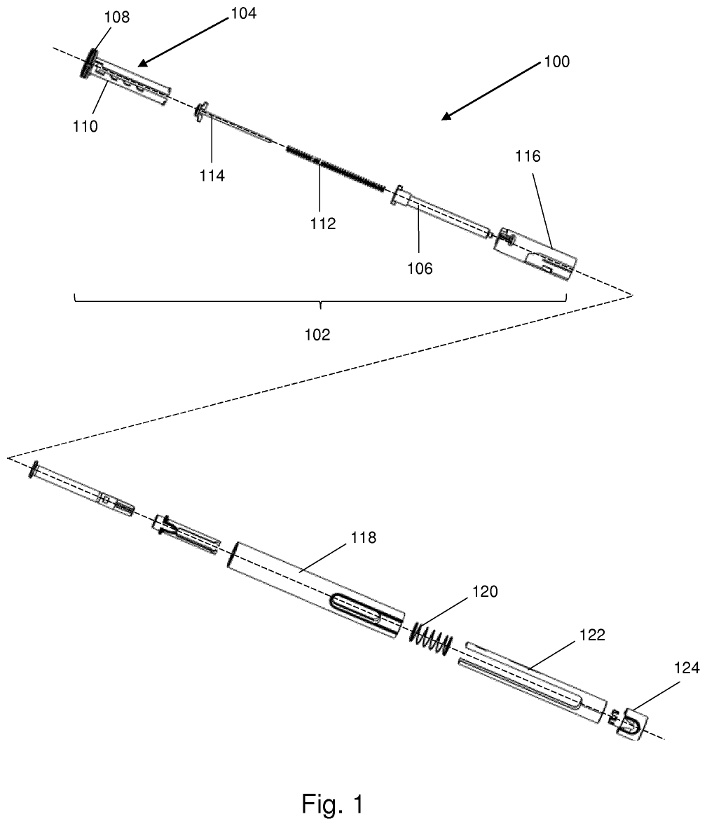

By way of introduction, an exemplary auto-injector 100, of a type which provides for both automatic needle insertion and automatic fluid delivery, will now be described by way of introduction with reference to FIGS. 1 to 3.

FIG. 1 shows an exploded view of the exemplary auto-injector 100. The auto-injector 100 comprises a firing assembly 102. The firing assembly comprises a rear cap 104 and a plunger 106. The rear cap 104 comprises a head 108 and an elongate member 110. The rear cap 104 and the plunger 106 are connected to each other such that, before firing, relative axial movement between them is resisted or prevented. The connection between the rear cap 104 and the plunger 106 is releasable such that after activation of the auto-injector 100, relative axial movement between them is permitted. The nature of the releasable connection is discussed in more detail below.

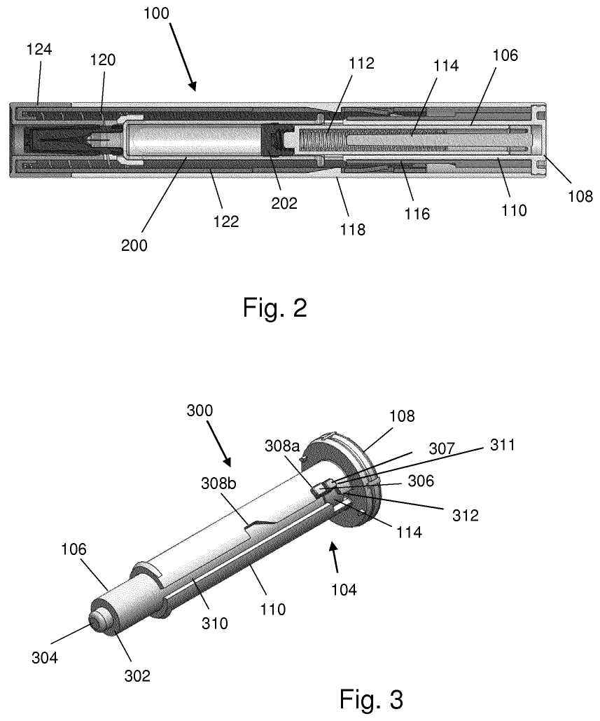

The firing assembly 102 also comprises a biasing member 112 for driving the plunger 106 axially forwards and into a barrel of a syringe (shown in FIG. 2) retained within the auto-injector 100. In one example the biasing member 112 is a drive spring (e.g. a compression spring) and will be referred to as such throughout, although this should not be construed as limiting and the skilled person will appreciate that other means may be used to drive the plunger forwards.

In the example of FIG. 1, the plunger 106 is telescopically received within the elongate member 110 of the rear cap 104. The drive spring 112 is positioned between the rear cap 104 and the plunger 106 such that they are biased in opposite axial directions relative to each other. This is best shown in FIG. 2, which is a section through an auto-injector 100 in an assembled state before activation and with a syringe 200 retained therein. The plunger 106 is received within the elongate member 110. The plunger 106 is a hollow tube with an open end at the rear and the drive spring 112 is received within the plunger 106. A first end of the drive spring 112 abuts a forward end of the plunger 106 and a second end of the drive spring 112 is fixed with respect to the rear cap at least during delivery of a medicament from the syringe. In the example of FIG. 2, the drive spring 112 is coupled to an end of dose indicator 114, which in turn is coupled to the rear cap 104. The end of dose indicator provides a reaction component against which the drive spring 112 reacts. Expansion of the drive spring 112 drives the plunger 106 forwards into the barrel of the syringe 200. In FIG. 2, a forward end of the plunger 106 is shown abutting a bung 202.

The auto-injector 100 also comprises a clutch 116, which is positioned around the elongate member 110. Before activation of the auto-injector 100, the clutch 116 is rotationally coupled to the plunger 106 and to the end of dose indicator 114. Rotation of the clutch 116 therefore causes rotation of the plunger 106 and the end of dose indicator 114. As explained below, on activation of the auto-injector 100, the clutch 116 rotates, thereby rotating the plunger 106 relative to the rear cap 104 to release the connection therebetween. Operation of the clutch 116, and of the end of dose indicator 114, is explained in more detail below.

The auto-injector 100 also comprises a main body 118, which houses the firing mechanism 102, the syringe 200 and other features necessary for operation of the auto-injector 100. The main body 118 may comprise a plurality of separate parts. The main body 118 comprises a syringe locator, which in exemplary arrangements comprises one or more features for receiving and optionally retaining a syringe in position within the main body 118.

The auto-injector also comprises a lockout spring 120 and lockout shroud 122, wherein the lockout spring 120 is configured on release thereof to displace the lockout shroud 122 axially forwards to cover a needle of the syringe. A cap 124 also forms part of the auto-injector and covers a needle or forward end of the auto-injector prior to use.

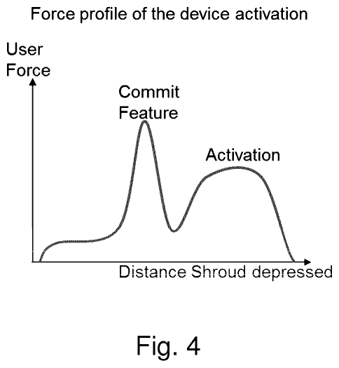

FIG. 3 shows a perspective view of an assembly 300 for a firing mechanism (absent the clutch 116). The assembly 300 comprises the rear cap 104 and the plunger 106. The plunger 106 is telescopically received within the elongate member 110. The assembly 300 also comprises a plunger driver to drive the plunger 106 axially forwards, which in the exemplary arrangements disclosed herein comprises a compression spring 112 although the skilled person will understand that other arrangements are possible.

The exemplary plunger 106 comprises a cylindrical tube that is open at a rear end and closed at a forward end. The forward end of the plunger 106 comprises a shoulder 302 and a projection 304 configured to engage a bung 202 in a syringe barrel. The plunger 106 also comprises a lug 306 configured to engage with any of a plurality of recesses 308a, 308b in the elongate member 110. In the exemplary arrangements of FIG. 3 the lug 306 extends radially from an outer surface of the plunger 106.

The elongate member 110 comprises an axial channel 310. The plurality of recesses 308a, 308b are formed in a sidewall of the channel 310. That is, the plurality of recesses 308a, 308b extend circumferentially (or transverse to the axial channel) around the outer wall of the elongate member 110. It is noted that while only two recesses 308a, 308b are shown in FIG. 3, more recesses may be provided in the elongate member 110. The recess 308b comprises an angled rear surface and a front surface that is perpendicular to an axial direction (or longitudinal axis) of the auto-injector 100. The recesses 308a, 308b are configured to receive the lug 306 of the plunger 106. FIG. 3 shows the lug 306 received in a rearward recess 308a.

The channel 310 and the recesses 308a, 308b are configured such that rotation of the plunger 106 relative to the elongate member 110 in a first direction moves the lug 306 into the recesses 308a, 308b and rotation in a second, opposite direction moves the lug 306 out of the recesses 308a, 308b.

The plunger 106 and the rear cap 104, in particular the elongate member 110, define an axial length of the assembly 300. The axial length of the assembly 300 determines a start position of the forward end of the plunger 106 before release of the connection of the plunger 106 and the elongate member 110.

During assembly, the plunger 106 is connected to the elongate member 110 at any of a plurality of positions on the elongate member 110 and/or the plunger 106 to alter a combined axial length of the plunger 106 and the elongate member 110. The connection may be made directly or indirectly through a plunger carrier. In the example described here the connection is direct. The plunger 106 may be received within the elongate member 110 such that the lug 306 is in the channel 310. The plunger 106 may then be displaced relative to the elongate member 110 until the lug 306 is aligned with one of the recesses 308a, 308b. The plunger 106 may then be rotated such that the lug is received within the one of the recesses 308a, 308b with which it was aligned. The force provided by the drive spring 112 retains the lug 306 within the recess 308a, 308b against the forward surface thereof.

FIG. 3 further illustrates a rear end of the end of dose indicator 114 which includes a lug 307 projecting radially outward. The lug 307 engages with the recess 308a formed in the sidewall of the channel 310, immediately behind the lug 306 of the plunger 106.

The recess 308a presents a rear stop surface 311 extending in a generally circumferential direction. The circumferential extent of the stop surface 311 is such that combined rotation of the plunger 106 and end of dose indicator 114 to a position such that the plunger lug 306 is located within the channel 310 results in the continued blocking of the lug 307 (preventing rearward movement of the end of dose indicator 114). However, further rotation of the end of dose indicator 114, as will be described below, takes the lug 307 past the end of the stop surface 311 and aligns it with an effective upper extension 312 of the channel 310.

During assembly of the auto-injector, the assembly comprising the plunger, the end of dose indicator and the rear cap is set using any of the methods and apparatus described herein. The plunger and end of dose indicator are connected to the elongate member of the rear cap. The connection is releasable in that, upon activation of the auto-injector, the connection is released to allow relative axial movement of the plunger and the end of dose indicator. Also, the connection may be made at any of a plurality of positions on the elongate member or the plunger. That is, one or both of the elongate member and the plunger may be configured to have a plurality of locations at which the connection may be made. The lug 307 of the end of dose indicator 114 is always engaged with the rearmost recess 304a regardless of the initial position of the plunger.

Accordingly, the combined axial length of the plunger and the rear cap is set to the desired length based on a fill volume (or bung position) of a syringe that the auto-injector is intended for use with. In so doing, the gap between the bung of the syringe, which sits at a position in the barrel that is dependent on the fill volume, is controlled. That is, if the combined axial length of the plunger and the rear cap is extended then the auto-injector may be used for syringes having a smaller fill volume, or otherwise having a bung that is initially positioned further forwards within the barrel, e.g. if the barrel is of a greater diameter but the fill volume remains the same. The start position of a forward end of the plunger is adjusted during assembly.

Once assembled, the user has no control over the combined length of the rear cap and the plunger. Operation of the auto-injector 100 is described below using the reference numerals of the exemplary arrangement shown in FIGS. 1 to 3.

In use, a user removes the cap 124 of the auto-injector 100, which in turn removes a rigid needle shield covering the needle. Removal of the cap also exposes the lockout shroud 122, which protrudes from a forward end of the body 118. The user places a forward end of the lockout shroud 122 against an injection site and pushes the auto-injector 100 forwards onto the injection site. This action pushes the lockout shroud 122 rearwards within the auto-injector 100. The lockout shroud interacts with the clutch 116 to rotate it. This may be done by forcing a surface (or pip) of the lockout shroud 122 against an angled surface on the clutch 116, which translates the rearward motion of the lockout shroud 122 into rotational motion of the clutch 116.

As the clutch 116 is rotationally coupled to the plunger 106 and to the end of dose indicator 114, rotation of the clutch 116 causes rotation of the plunger 106 and the end of dose indicator 114. In some arrangements, the clutch 116 may have an internal track located on an internal wall thereof and that receives a lug of the plunger 106 and a lug of the end of dose indicator 114. These lugs may be the same as the lugs 306, 307 described with reference to FIG. 3. Rotation of the plunger 106 with respect to the rear cap 104 releases the connection between the rear cap 104 and the plunger 106, allowing the plunger 106 to be driven forwards under force of the drive spring 112. In the examples of FIGS. 1 to 3, this is provided by rotating the lug 306 of the plunger rod 106 out of the recess 308a and into the axial channel 310. The lug 306 is thereby allowed to travel forwards within the channel 310. In this state, the lug 307 of the end of dose indicator 314 has been rotated to the same extent.

The drive spring 112 then acts against the plunger 106 and the rear cap 104 via the end of dose indicator 114 which is prevented from moving rearwards by the stop surface 311. Because the rear cap 104 is fixed within the auto-injector 100, the force delivered by the drive spring 112 acts to drive the plunger 106 into the barrel of the syringe. Because the gap between the forward end of the plunger 106 and the bung 202 has been controlled during assembly, the plunger 106 does not accelerate above a safe velocity that would risk damage to the syringe 200 or harm or discomfort to the subject of the injection.

The force applied by the drive spring 112 acting against the bung initially moves the entire syringe forwards through the device body. This causes insertion of the needle into the skin. At a position defined to provide an optimal needle insertion depth, the syringe body bottoms out within the body to prevent further forward movement of the syringe and the needle. At this point the force exerted by the drive spring causes the bung to move forwards through the syringe body causing fluid to be injected through the needle.

The following sequence of steps then occurs:

(a) The plunger 106 is driven forward to a position at which the lug 306 of the plunger meets a rotation ramp (not shown) provided on an inner surface of the clutch 116. As the lug 306 remains confined within the channel 310 of the elongate member 110, and therefore cannot rotate relative to the elongate member 110, the lug 306 causes the clutch 116 to rotate (counter-clockwise when viewed from the rear end of the device). Rotation of the clutch 116 by the lug 306 in turn rotates the end of dose indicator 114 (again in a counter-clockwise direction) due to engagement of the clutch with the lug 307.
(b) The lug 307 is moved across the stop surface 311 until the lug is free to move rearward into the channel extension 312. Part of the stop surface 311 may be slightly angled to encourage rotation of the lug 307 across the stop surface.
(c) As the lug 307 is now free to move rearwardly along the channel extension 312, the end of dose indicator 114 is forced rearwards by the force exerted by the drive spring 112 until an end of the end of dose indicator strikes an inner surface of the head 108 of the rear cap. This impact results in an audible sound or click.
(d) The plunger continues to move through the device body with the lug 306 remaining confined within the channel 310 until the plunger bottoms out.
(e) The user then removes the needle from the skin. The lockout spring 120 pushes the lockout shroud 122 forward to re-cover the needle. Although not shown in the Figures, snap features may be provided between the lockout shroud 122 and the main body 118 to prevent the lockout shroud from being pressed back into the body.

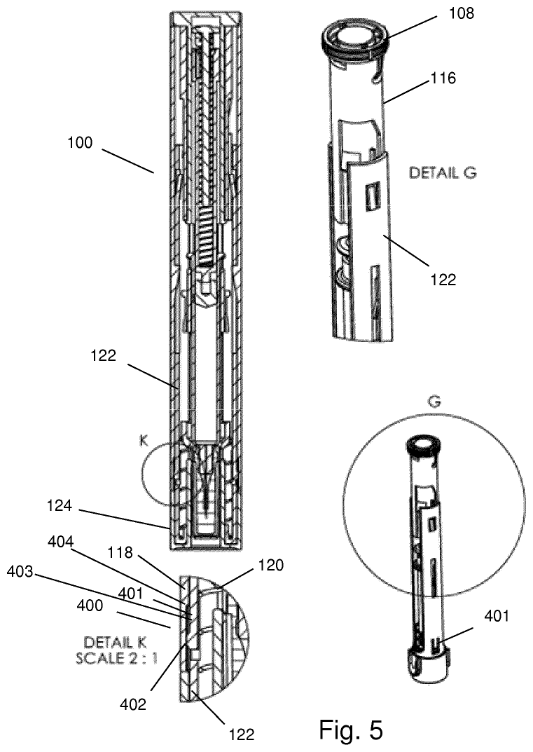

The auto-injector described above presents a resistance to activation that is primarily defined by the lockout spring 120. It is the force exerted by this spring that is overcome when the user presses the lockout shroud against the skin. Any additional force required to rotate the clutch and release the plunger is relatively small. As already noted above, such a configuration may cause the device to be activated accidentally when a user is performing site roaming, i.e. testing different areas of the skin to identify a comfortable injection site. It is therefore desirable to implement a commit feature that results in a user resistance force profile such as that illustrated in FIG. 4. With such a resistance force profile, a relatively large resistance force is encountered after the lockout shroud has been pressed into the device by a second predefined distance that is less than a first predefined distance that will activate the device.

The object of the commit feature is to generate a force that the user has to overcome to intentionally activate the device. Inadvertent activation is prevented (or at least the risk of such inadvertent activation mitigated) and the possibility for site roaming is enabled. The resistance force profile should have the following characteristic:
1. Relatively low force site roaming.
2. Relatively high force for the commit feature.
3. Lower force for the activation of the device, i.e. the user should not be able to perceive a difference between commitment and activation.

The skilled person will appreciate that a force resistance profile such as that shown in FIG. 4 can be implemented in many different ways. For example, features could be implemented between the lockout shroud and the main body to provide a force resistance at said second predefined distance.

FIGS. 5 to 8 illustrate an auto-injector substantially as described above with reference to FIGS. 1 to 3 but modified to implement a commit feature.

FIG. 5 illustrates the modified device in a stored state, with the front cap 124 attached to the main body, with detail G showing the lockout shroud 122 coupled to the firing mechanism including the clutch 116, with a head 108 of the rear cap visible behind the clutch. Detail K shows the implementation of a commit feature 400 implemented at a front end of the auto-injector between the main body 108 and the lockout shroud 122. Specifically, the commit feature 400 comprises a pair of flexible legs 401, one on each side of the lockout shroud 122, that provide a resilient component of the commit feature 400. Each flexible leg 401 comprises a lug 402 that projects radially outwards from the main inner surface of the lockout shroud. These lugs 402 are received within respective tracks 403 that extend longitudinally along an inner surface of the main body 108. Each track presents at its rearmost end a lip 404.

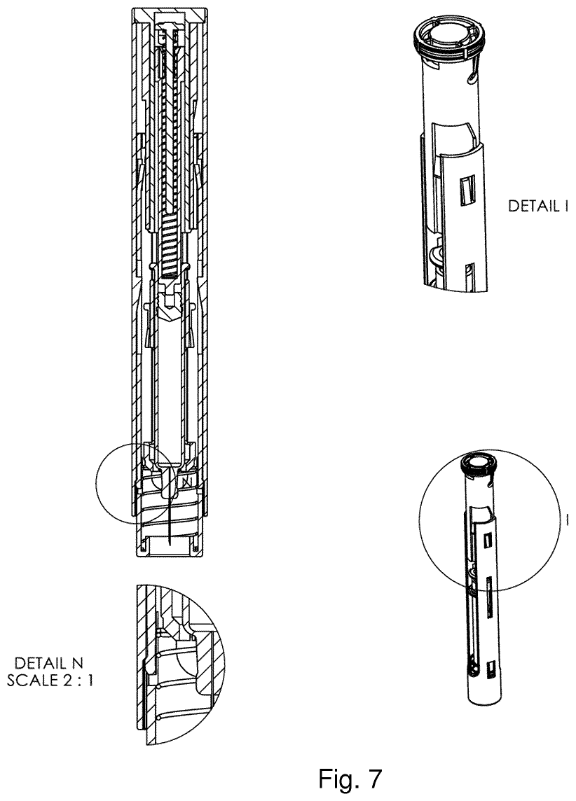

FIG. 6 illustrates the modified device with the front cap 124 removed, otherwise the state of the device remains unchanged from that shown in FIG. 5.

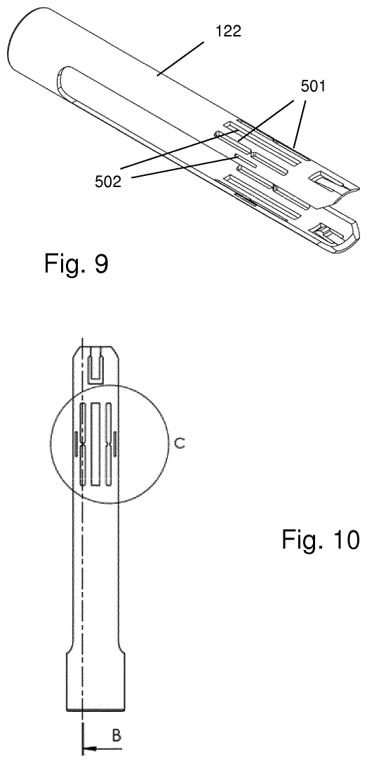

FIG. 7 illustrates the modified device in a state where a user has pushed the lockout shroud into the main body sufficient to engage the commit feature. Engagement of the lugs 402 with the rearmost end of the tracks 403 defines the second predefined distance mentioned above, i.e. the position at which the increased commit force resistance is encountered. In order to push the lockout shroud past this position a force sufficient to flex the lugs 402 inwards and over the end of the tracks 403 must be exerted by the use on the end of the lockout shroud. The cooperating shapes of the lugs 402 and the ends of the tracks 403 may be used to define the commit force resistance. Once the lugs are pushed over the ends of the tracks, the force required to move the shroud further into the main body is reduced until the shroud engages the clutch to rotate the clutch and activate the firing mechanism. FIG. 8 shows the device in a state where the first predefined position has been reached, the clutch rotated, and the device activated to insert the needle.

FIGS. 9 and 10 illustrates an alternative lockout shroud 122 configured to implement an alternative commit feature. In this embodiment the shroud 122 is provided on each side with a pair of guide slots 501. These guide slots have a reduced width at a centre point. Further slots 502 are provided on each side of the guide slots to allow the guide slots to flex to a small degree. Although not shown in the figures, the guide slots 501 are engaged by respective pips that extend inwardly from the main body 108. As the lockout shroud 122 is pushed into the device, at said second predefined position the pips will engage with the reduced width regions of the guide tracks, presenting an increased force resistance. As an increased force is applied to overcome the commit feature, the guide tracks will flex to allow the pips to proceed, until the first predefined position is reached and the device is activated.

It will be appreciated by the person of skill in the art that various modifications may be made to the above described embodiments without departing from the scope of the present invention.

1. An injection device comprising:
a body for receiving a syringe;
a firing mechanism comprising a plunger configured to be axially displaced in a forward direction within the body and a driver system for driving the plunger forwards upon activation of the injection device;
a lockout shroud telescopically coupled to a forward end of the body so that it projects from the forward end at least immediately prior to activation in order to shroud a syringe needle;
a biasing member for biasing the lockout shroud in a forward direction relative to the body, the lockout shroud being coupled to the firing mechanism such that a predefined rearward movement of the shroud relative to the body releases the driver system to drive the plunger forward; and
a lockout shroud inhibitor comprising a pair mechanically interfering features located on the lockout shroud and the body respectively, the pair of mechanically interfering features being configured to engage on the predefined rearward movement of the shroud to provide increased resistance to further rearward movement of the lockout shroud,
wherein the lockout shroud is configured to move rearwards relative to the body by a first distance to engage the mechanically interfering features at a predefined position, and wherein further rearward movement of the lockout shroud after the increased resistance is overcome releases the driver system.

2. An injection device according to claim 1, wherein said lockout shroud inhibitor provides a reduced resistance after said increased resistance is overcome and prior to activation.

3. An injection device according to claim 1, wherein said lockout shroud inhibitor acts between the lockout shroud and the body.

4. (canceled)

5. An injection device according to claim 4, wherein the pair of mechanically interfering features comprises a resilient member on one of the lockout shroud and the body and a lip on the other of the lockout shroud and the body, the resilient member engaging the lip at substantially said predefined position and being configured to flex over the lip when an increased force is applied between the lockout shroud and the body.

6. An injection device according to claim 4, wherein the pair of mechanically interfering features comprises a longitudinally extending track on one of the lockout shroud and the body and a feature for engaging and travelling along said track on the other of the lockout shroud and the body, said track comprising a restriction part-way along its length and having sides configured to flex in a transverse direction to enable said feature to pass the restriction when an increased force is applied between the lockout shroud and the body.

7. An injection device according to claim 1 and comprising a clutch having a substantially fixed axial position within the body and being coupled to the lockout shroud such that said first predefined movement of the lockout shroud rotates the clutch to thereby release the driver system.

8. An injection device according to claim 1, wherein said biasing member is a compression spring coupled between the lockout shroud and the body.

9. An injection device according to claim 1, wherein said lockout shroud is located partially within said body.
